(19) United States
(12) Patent Application Publication
(10) Pub. No.: US 2005/0288663 A1
(43) Pub. Date: Dec. 29, 2005

(54) Method and apparatus for substantial uniform ablation about a bipolar array of electrodes

(76) Inventor: Kamran Behzadian, Sunnyvale, CA (US)

Correspondence Address: InhouseIP Counsel, 280 Colorado Avenue, Palo Alto, CA, 94301, US

(21) Appl. No.: 10/878,168

(22) Filed: Jun. 28, 2004

(51) Int. Cl. A61B 18/14

(52) U.S. Cl. 606/41; 606/50; 606/48

(57) ABSTRACT

The described invention relates to a medical device for a substantially uniform ablation of animal or human tissue comprising of a bipolar generator for generating radio frequency at an electrode, a probe having a handle, and an elongated member. The elongated member has a proximal and distal end with an electrode cluster of three or more electrodes located on the distal end of the elongated member. The electrodes are electrically insulated from each other and at least two of the electrodes have dissimilar polarity from each other wherein at least one of the electrodes has a high voltage polarity and at least one of the electrodes has a return polarity. Also, during ablation, there is a polarity alternator for changing the polarity of the electrodes and a tip at the far end of the distal end suitable for insertion into tissue.

### FIELD

The invention relates generally to a method and apparatus for utilizing energy, such as radio frequency energy, in a multi electrode and bipolar fashion to treat defined volumes of animal or human tissue uniformly and more particularly have the ability to concentrate lesion formation around desired electrodes through the use of a member having multiple electrodes whose polarity of one or more electrodes is independently controlled.

### BACKGROUND

Methods for treating damaged animal or human tissue, such as those with benign and malignant tumors, have been developed and improved for many years. Recently, a new technique known as radio frequency ablation has been developed in order to treat damaged tissue by destroying its damaged cells plus the adjacent undamaged cells to prevent further spreading. The radio frequency energy causes the tissue to heat up to a high temperature, therefore breaking apart and killing the cells. The goal is to produce immediate effects on the tissue and rapid postoperative wound healing.

In a monopolar ablation system, a probe or catheter containing electrodes with high voltage polarity that releases electrical energy, is placed inside the body while an electrode pad with return polarity that receives the electrical energy is placed outside on the patient's skin. The result is greater amounts of electrical energy being dispersed throughout the patient's body, therefore destroying more areas of tissue than necessary and missing targeted areas.

Following the development of this device is a new system known as a bipolar ablation system. This improved device contains both the electrodes with high voltage and return polarity on the same probe that is placed inside the tissue, therefore eliminating the need for an external return electrode pad. The electric field is present more local to the ablated area and not throughout the body, but its use is still limited due to an irregular formed lesion 42 as shown in FIG. 5.

While technology has resulted in our ability to ablate damaged tissue, there is still an important need for a bipolar ablation system that heats a zone about the cluster of electrodes on the probe uniformly, therefore producing a substantially uniform ablation lesion. The present invention provides such methods that will in turn enable practitioners to ablate a significantly greater amount of targeted areas of tissue combined with avoiding the unnecessary destruction of other healthy areas.

### SUMMARY

The invention described is a medical device for substantially uniform ablation of animal or human tissue comprising of a bipolar generator for generating radio frequency at an electrode, a probe having a handle, a tip tapered to provide a sharp point, and an elongated member. The elongated member has a proximal end and a distal end. At the distal end there is an electrode cluster of three or more electrodes wherein the electrodes are electrically insulated from each other and at least two of the electrodes have dissimilar polarity from each other. At least one of the electrodes has a high voltage polarity and at least one of the electrodes has a return polarity.

Optionally, during ablation a polarity alternator is used for changing the polarity of the electrodes. A tip at the far end of the distal end is suitable for insertion into tissue. The electrodes are optionally spaced evenly or optionally unevenly along the length of the distal end of the elongated member, such that the electrodes have dissimilar surface areas exposed to the tissue.

The electrodes are optionally composed of electrically conductive matter including copper, stainless steel, or precious metal plated material. They are preferably wrapped about the circumference of the elongated member and optionally shaped in the form of a coil. The coil electrodes optionally have a rectangular cross-section. Also, bends can be found anywhere along the length of the electrode cluster. For these reasons, the elongated member has varying flexibility along the length of the elongated member, thus allowing the desired ablation formation.

The described invention consists of a method for substantially uniform ablation of animal or human tissue further comprising the steps of a placement of an electrode cluster of three or more electrodes on the distal end of an elongated member adjacent to the tissue. The electrodes are capable of carrying an electrical charge.

Optionally, the method activates a bipolar generator to create dissimilar is polarity between the three or more electrodes such that the total surface area of the electrodes with a high voltage polarity is unequal to the total surface area of the electrodes having a return polarity, creating a higher current density about the electrodes with a lesser total surface area sufficient to cause ablation of tissue adjacent to the area of higher current density.

Optionally, the polarity of the three or more electrodes is altered individually to create a higher current density about the electrodes having a lesser surface area at a different point along the length of the cluster of electrodes prior to altering the polarity of electrodes, to ablate tissue adjacent to the area of higher current density to achieve uniform ablation of the tissue along the length of the cluster of electrodes. The alteration of the polarity of the three or more electrodes will optionally change individually from high voltage to return polarity, neutral, or remain at high voltage polarity and from return to high voltage polarity, neutral, or remain at return polarity. Lastly, as an option, the alteration of the polarity of the three or more electrodes which creates a higher current density about the electrodes having a lesser surface area at a different point along the length of the cluster of electrodes prior to altering the polarity of electrodes, to ablate tissue adjacent to the area of higher current density along the length of the cluster of electrodes is repeated as needed to achieve substantial uniform ablation of the targeted tissue.

### BRIEF DESCRIPTION OF DRAWINGS

FIG. 1 shows a side block view of a bipolar radio frequency ablation system-Bipolar Generator and Probe.

FIG. 2 shows a bipolar probe with an electrode cluster at the distal tip.

FIGS. 3A, 3B, and 3C show side views of a three electrode clusters with individual electrodes energized with different polarities and resulting electric field and lesion concentration zones.

FIGS. 4A and 4B show an embodiment with flexible coils with rectangular cross section coils with straight edges and a cut away view respectively.

FIG. 5 shows an irregular formed lesion.

FIG. 6 shows a substantially uniform ablated tissue.

FIG. 7 shows an embodiment of the device of the invention with 3 electrodes in the cluster of electrodes.

FIG. 8 shows insertion of the elongated member into a protein medium.

FIG. 9 shows the activation of the center electrode as the one with the lowest surface area and high current area.

FIG. 10 shows the activation of the most distal electrode as the one with the lowest surface area and high current area.

FIG. 11 shows the activation of the most proximal electrode as the one with the lowest surface area and high current area.

### DETAILED DESCRIPTION

This invention relates to a bipolar radio frequency ablation system and method. Radio frequency ablation may be performed through an open incision or through laparoscopy, which is performed through multiple small incisions, or percutaneously as required. The duration and power requirements of a radio frequency ablation procedure may depend on many factors, including the number of desired applications and location of the animal or human tissue being treated.

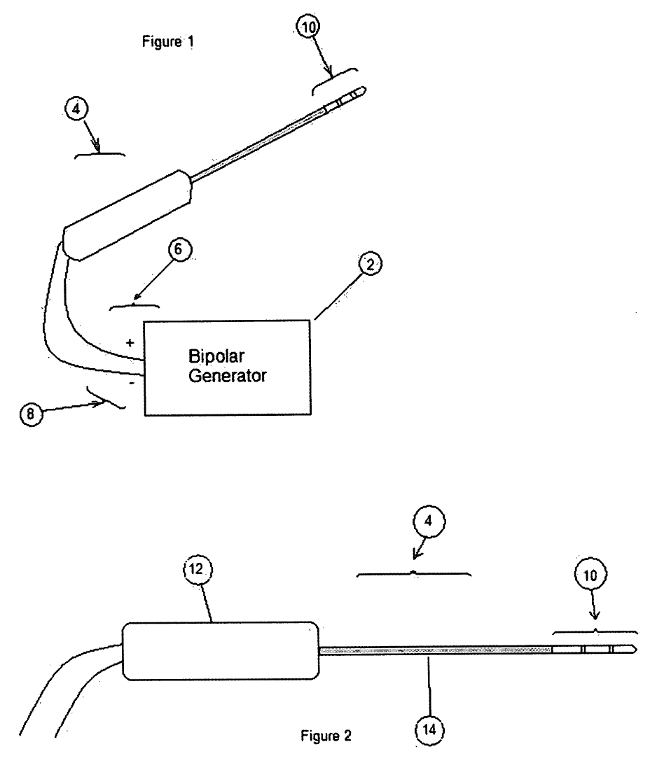

FIG. 1 shows a bipolar ablation system of the invention comprised of a bipolar generator 2 and a bipolar probe 4 with an electrode cluster that consists of three or more electrodes at the distal tip 10. The bipolar generator 2 may be a conventional general purpose electrosurgical power supply operating at a frequency in the range from about 400 kHz to about 1.2 MHz, with a conventional sinusoidal or non-sinusoidal wave form. The bipolar generator 2 has a positively charged 6 high voltage polarity and a negatively charged 8 return polarity. Such power supplies are available from many commercial suppliers. Additionally, the bipolar generator 2 is capable of altering individual electrodes from high voltage polarity to return polarity or neutral and from return polarity to high voltage polarity or neutral, also defined as a polarity alternator, based on temperature, current, voltage, or impedance feedback from the probe or on an activation time basis.

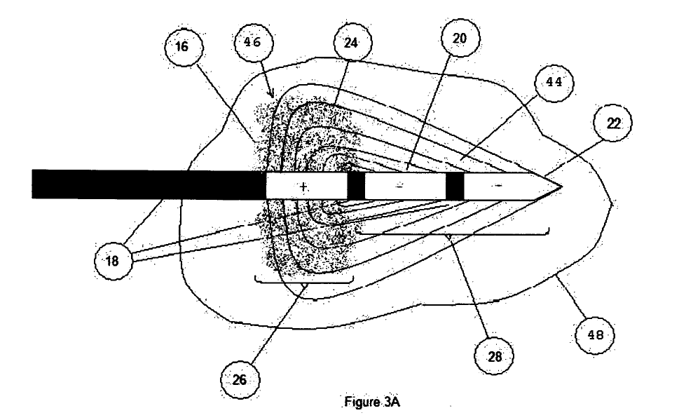

The bipolar probe 4, as shown in FIG. 2 is comprised of a handle 12, an elongated member 14, and an electrode cluster with three or more electrodes 10. The elongated member 14 is flexible. The flexible elongated member 14 is deflectable, allowing the creation of lesions in various shapes as needed to complete the procedure effectively.

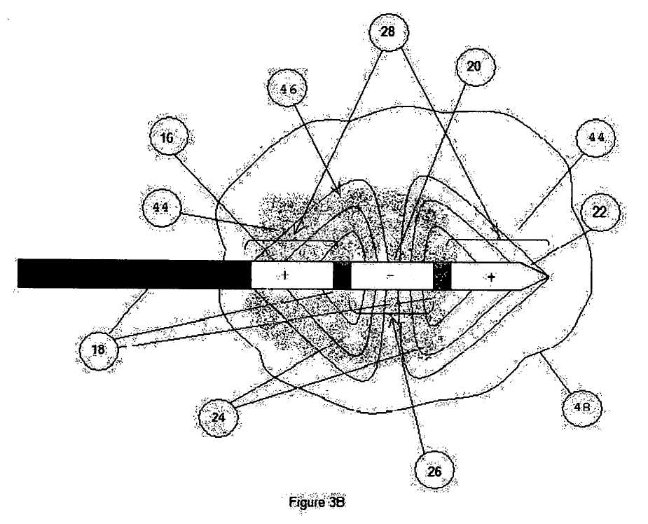

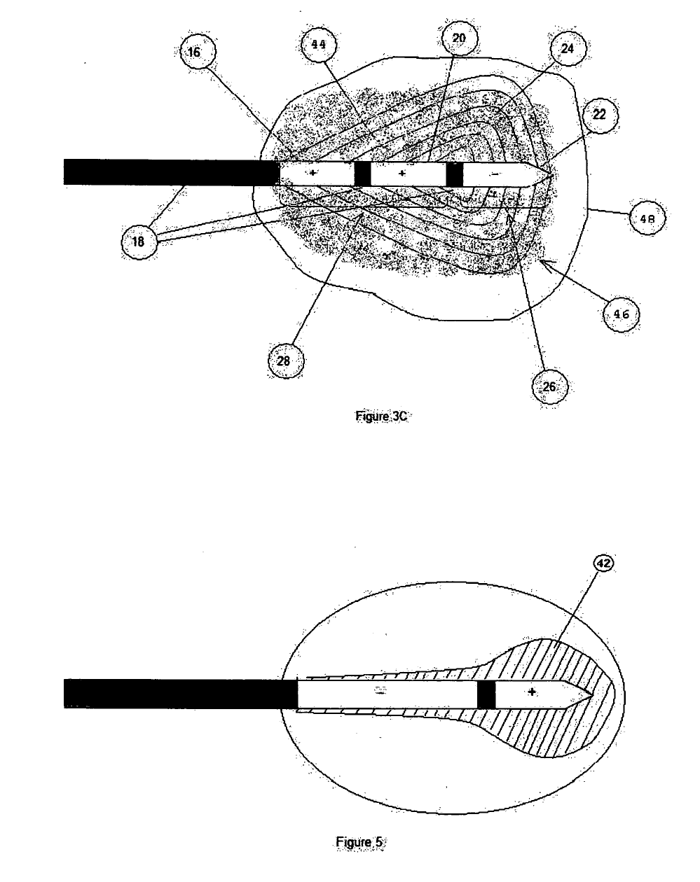

FIGS. 3A through 3C are examples of steps of ablating tissue by means of a bipolar probe 4 with an electrode cluster that consists of three or more electrodes at the distal tip 10 and with a resulting electric field when electrodes are identical in composition and geometry that will ultimately result in a substantial uniform lesion.

FIG. 3A shows dissimilar polarity by electrode 16 being energized with high voltage polarity, which is positively charged 6 and denoted by a “+” sign, and electrodes 20 and 22 being energized with return polarity, which is negatively charged 8 and denoted by a “−” sign. Item 18 are electrical insulation and can be found between the electrodes and on the proximal end of the probe. Dissimilar surface areas, shown with smaller surface area 26 and larger surface area 28, allows the electric field and therefore electrical current density to be higher 24 about electrode 16 and lower 44 about electrodes 20 and 22, therefore producing a higher current density 24 and lower current density 44, thus ablation heat generation is greatest about electrode 16 resulting in lesion generation 46 in targeted tissue 48 about electrode 16. The electrodes may be spaced evenly or unevenly with electrical insulation 18 found in between each electrode to achieve desired ablation.

Next step is represented in FIG. 3B. FIG. 3B shows dissimilar polarity by electrode 20 being energized with return polarity and electrodes 16 and 22 being energized with high voltage polarity. Item 18 are electrical insulation. Dissimilar surface areas allow the electric field and therefore electric current density to be higher 24 about electrode 20 and lower 44 about electrodes 16 and 22, therefore producing a higher current density 24 and lower current density 44, thus ablation heat generation is greatest about electrode 20 resulting in lesion generation 46 in targeted tissue 48 about electrode 20. The electrodes may be spaced evenly or unevenly with electrical insulation 18 found in between each electrode to achieve desired ablation.

Next step is represented in FIG. 3C. FIG. 3C shows dissimilar polarity by electrode 22 being energized with return polarity and electrodes 16 and 20 being energized with high voltage polarity. Item 18 are electrical insulation. Dissimilar surface areas allow the electric field and therefore electric current density to be higher 24 about electrode 22 and lower 44 about electrodes 16 and 20, therefore producing a higher current density 24 and lower current density 44, thus ablation heat generation is greatest about electrode 22 resulting in lesion generation 46 in targeted tissue 48 about electrode 22. The electrodes may be spaced evenly or unevenly with electrical insulation 18 found in between each electrode to achieve desired ablation.

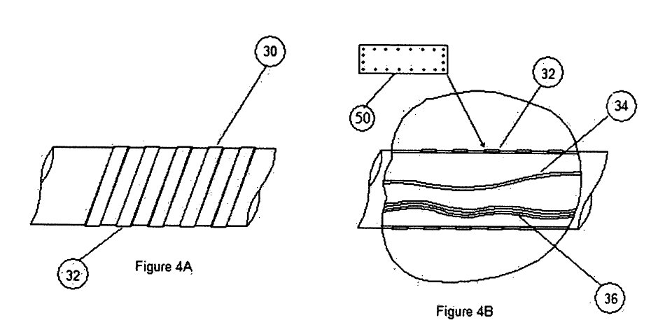

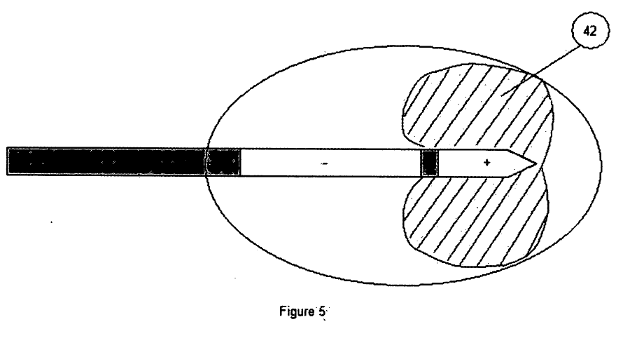

FIG. 4A shows the electrodes wrapped about the circumference of the elongated member 14 in the form of a flexible coil 30. A higher current density 24 is present about the straight edges 32.

FIG. 4B shows a rectangular cross section 50 of the flexible coils 30 with straight edges 32 and temperature feedback wires 34 and 36 for individual electrodes. Therefore, an electrode shaped in the form of flexible coils 30 with straight edges 32 wrapped about the circumference of the elongated member 14 with rectangular cross section 50 will produce more uniform ablations over the length of the electrodes. An electrode made up of many straight edges 32 spaced closely to each other produces a substantial uniform ablation 40 that is made up of small lesions about the straight edges 32 that are spaced closely to each other that propagate and join into a large and substantial uniform ablation 40 about the entire length of the electrode.

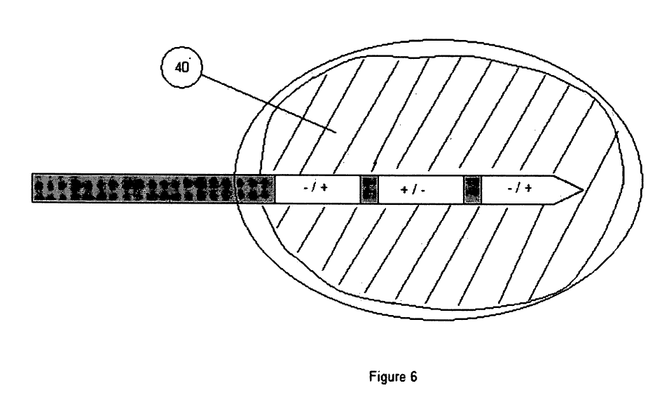

FIG. 6 shows a uniformly heated zone around a bipolar array of electrodes by activating a bipolar generator 2 to create dissimilar polarity between the electrode cluster or three or more electrodes 10 with dissimilar surface areas such that the total surface area of the electrodes with a high voltage polarity is unequal to the total surface area of the electrodes having a return polarity, creating a higher current density 24 about the electrodes with a lesser surface area 26 sufficient to cause ablation tissue adjacent to the area of higher current density 24 and then a polarity alternator created by altering the polarity of the electrode cluster of three or more electrodes 10 to create a higher current density 24 about the electrodes having a lesser surface area 26 at a different point along the length and therefore achieving a substantial uniform ablation 40 of the tissue along the length of the electrode cluster.

Current density is independent of polarity, therefore any electrodes with high voltage polarity or return polarity in FIGS. 3A through 3C may have their polarities altered in order to shift high current density 24 to the tissue surrounding a different electrode.

The ability to control lesion formation about specific electrodes in a bipolar device enables the device to produce a substantial uniform ablation 40 by methodically manipulating current densities about electrodes in an array of electrodes and therefore creates a substantial uniform ablation of the tissue along the electrode cluster.

A substantial uniform ablation 40 may be verified by monitoring lesion temperature by locating feedback temperature sensors under electrodes, or monitoring electrical current, voltage, impedance, or per a time and power input protocol embedded in the generator. Substantial uniform ablations 40 treat the material portions of tissue that is essential and adequate for destroying the damaged and the adjacent undamaged cells.

### EXAMPLE

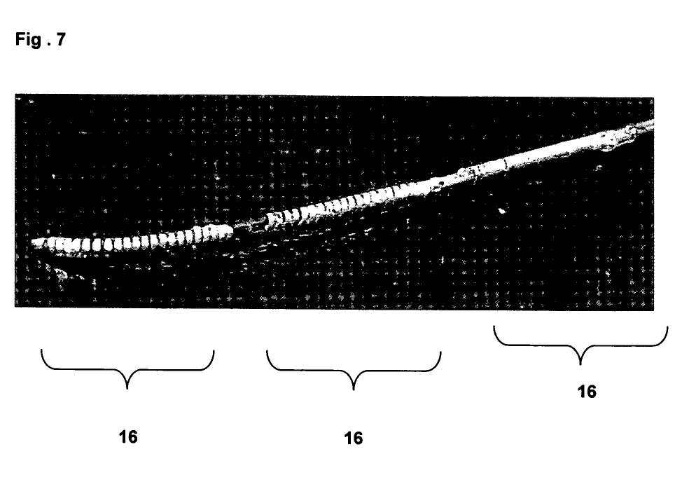

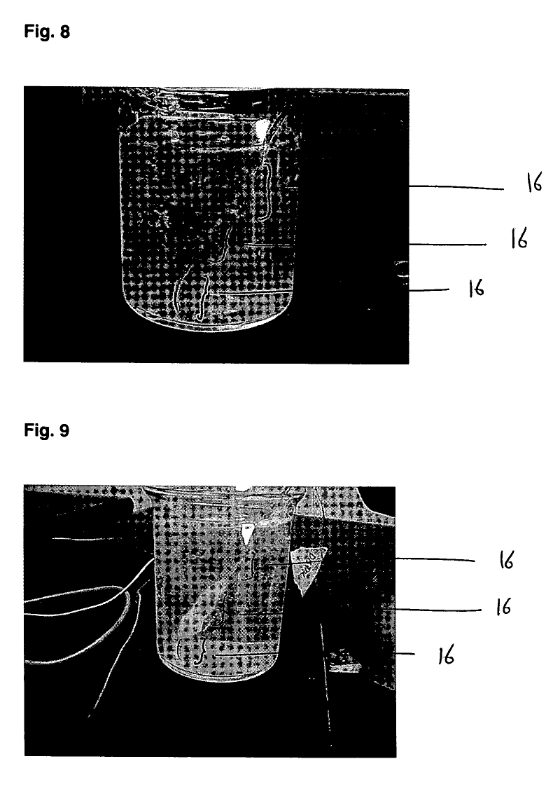

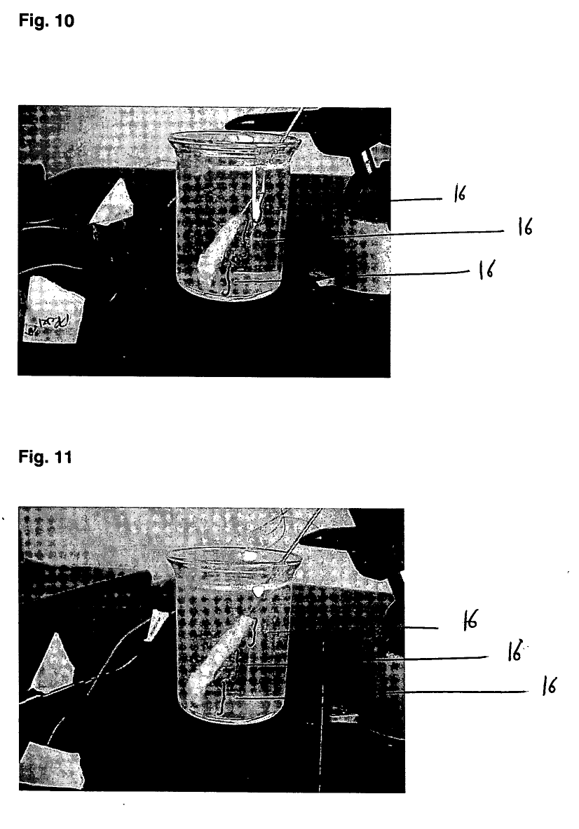

A transparent protein medium, such as a collagen solution or egg white mixed with water, is brought to approximately 37+ C. in a water bath. In this example, a homogeneous egg white solution was used. Thereafter, the bipolar ablation device was inserted into the medium. The lesions are made due to and are contained within the electric field per current densities present in the electric field created about the electrodes. Therefore, shape of electric field and therefore lesions shape is independent of medium and lesions shaped produces in this medium are identical to lesions made in any tissue or reasonable medium. FIG. 7 shows an embodiment of the invention with a three (3)-element bipolar ablation device. Coiled electrode elements with straight edged cross section are used in this optional embodiment. The elongated member is inserted into a protein based transparent medium in FIG. 8. In FIG. 9, the central electrode is energized with return polarity with negative charge and the distal and proximal electrodes are energized with high voltage polarity with positive charge. This causes increased heat surrouding the center electrode which causes the hardening of the egg white demonstarating the effectiveness of the device of the invention in formin a lesion about the center electrode due to the presence of high current density about the middle electrode. In FIG. 10, after alteration of the charge in the electrodes, the distal electrode is now energized with return polarity with negative charge while the middle and proximal electrodes are energized with high voltage polarity with positive charges. The area immediately surrounding the most distal electrode is heated which results in the hardning of the egg white surrounding the distal electrode. If inserted into a tissue, a lesion would be formed about the distal electrode due to the presence of high current density about the distal electrode.

In FIG. 11, after altering the charge of the electrodes, the proximal electrode is energized with return polarity with negative charge while the distal and middle electrodes are energized with high voltage polarity with positive charges. This demonstrates the formation of a lesion about the proximal electrode due to the presence of high current density about the proximal electrode.

From the aforementioned description, it is appreciated how the objectives and features of the above-described invention are met. First, the invention provides a minimally invasive surgical tool and technique to treat tissue that may be cancerous. Second, completion of lesion formation about the electrode elements will produce a substantial uniform lesion about the elongated member of the invention. It is appreciated that various modifications of the apparatus and method are possible without departing from the invention, which is defined by the claims set forth below.

1. A medical device for substantially uniform ablation of animal or human tissue comprising:
a. a bipolar generator for generating radio frequency at an electrode;
b. a probe having a handle and an elongated member;
c. the elongated member having a proximal and a distal end;
d. an electrode cluster of three or more electrodes located on the distal end of the elongated member, the electrodes electrically insulated from each other and at least two of the electrodes having dissimilar polarity from each other, at least one of the electrodes having a high voltage polarity and at least one of the electrodes having a return polarity, during ablation.
e. a polarity alternator for changing the polarity of the electrodes during ablation;
f. and a tip at the far end of the distal end suitable for insertion into tissue, wherein alteration of the polarity of the three or more electrodes will change individually from high voltage to return polarity, neutral, or remain at high voltage polarity and from return to high voltage polarity, neutral, or remain at return polarity.

2. The medical device of claim 1 wherein the electrodes are spaced evenly along the length of the distal end of the elongated member.

3. The medical device of claim 1 wherein the electrodes are spaced unevenly along the length of the distal end of the elongated member.

4. The medical device of claim 1 wherein the elongated member is flexible.

5. The medical device of claim 1 wherein the electrodes have dissimilar surface areas exposed to the tissue.

6. The medical device of claim 1 wherein the tip is tapered to provide a sharp point.

7. The medical device of claim 1 wherein the electrodes are composed of matter selected from the group consisting of lead, stainless steel, or gold-plated material.

8. The medical device of claim 1 wherein the electrodes are shaped in the form of a coil wrapped about the circumference of the elongated member.

9. The medical device of claim 8 wherein coil electrodes have a rectangular cross-section.

10. The medical device of claim 8 wherein the form of a coil wrapped about the circumference of the elongated member allows flexibility of the elongated member.

11. The medical device of claim 1 having a bend anywhere along the length of the electrode cluster.

12. The medical device of claim 1 wherein the elongated member has varying flexibility along the length.

13. A method for substantially uniform ablation of animal or human tissue further comprising the steps of:
a. placement of an electrode cluster of three or more electrodes on the distal end of an elongated member adjacent to the tissue, the electrodes capable of carrying an electrical charge,
b. activating a bipolar generator to create dissimilar polarity between the three or more electrodes such that the total surface area of the electrodes with a high voltage polarity is unequal to the total surface area of the electrodes having a return polarity, creating a higher current density about the electrodes with a lesser total surface area sufficient to cause ablation of tissue adjacent to the area of higher current density,
c. altering the polarity of the three or more electrodes individually to create a higher current density about the electrodes having a lesser surface area at a different point along the length of the cluster of electrodes prior to altering the polarity of electrodes, to ablate tissue adjacent to the area of higher current density to achieve uniform ablation of the tissue along the length of the cluster of electrodes, wherein the alteration of the polarity of the three or more electrodes will change individually from high voltage to return polarity, neutral, or remain at high voltage polarity and from return to high voltage polarity, neutral, or remain at return polarity.

14. (canceled)

15. Method in claim 13 wherein the alteration of the polarity of the three or more electrodes individually to create a higher current density about the electrodes having a lesser surface area at a different point along the length of the cluster of electrodes prior to altering the polarity of electrodes, to ablate tissue adjacent to the area of higher current density along the length of the cluster of electrodes is repeated as needed to achieve substantial uniform ablation of the targeted tissue.
